(19) United States
(12) Patent Application Publication
(10) Pub. No.: US 2018/0296818 A1
(43) Pub. Date: Oct. 18, 2018

(54) Protected Medical Valve

(71) Applicants: Eric P. Chiang, San Antonio, TX (US); Eric Haddad, Boerne, TX (US)

(72) Inventors: Eric P. Chiang, San Antonio, TX (US); Eric Haddad, Boerne, TX (US)

(21) Appl. No.: 16/002,134

(22) Filed: Jun. 7, 2018

Related U.S. Application Data
(63) Continuation-in-part of application No. 15/486,254, filed on Apr. 12, 2017.

(51) Int. Cl. A61M 39/12; A61M 39/26

(52) U.S. Cl. CPC A61M 39/12; A61M 2039/1072; A61M 2205/0205; A61M 39/26

(57) ABSTRACT

An apparatus for protecting a medical valve are disclosed, including a housing having a proximal female luer connection, a valve within the proximal female luer connection, a distal male luer tip, and a fluid passageway between the proximal female luer connection and the distal male luer tip; and a cover member comprising a substantially hollow cylinder with two or more flaps at a proximal end and an aperture at the distal end; wherein the aperture of the cover member is configured to receive the housing so that the two or more flaps are positioned above the proximal female luer connection; and wherein the two or more flaps are configured to fold into the substantially hollow cylinder when pressed on by a syringe or an IV in order to allow the connection of the syringe or the IV onto the proximal female luer connection. Other embodiments are described and claimed.

### I. CROSS REFERENCE TO RELATED APPLICATIONS

The application is a continuation-in-part application of U.S. patent application Ser. No. 15/486,254, titled “Protected Medical Valve”, filed Apr. 12, 2017, the contents of which are hereby incorporated by reference.

### II. BACKGROUND

The invention relates generally to the field of injection ports of liquid or gas delivery systems used in the medical setting. More particularly, the invention relates to a method and apparatus for covering and protecting medical connector devices.

### III. SUMMARY

In one respect, disclosed is an apparatus comprising: a housing having a proximal female luer connection, a valve within the proximal female luer connection, a distal male luer tip, and a fluid passageway between the proximal female luer connection and the distal male luer tip; and a cover member comprising a substantially hollow cylinder with two or more flaps at a proximal end and an aperture at the distal end; wherein the aperture of the cover member is configured to receive the housing so that the two or more flaps are positioned above the proximal female luer connection; and wherein the two or more flaps are configured to fold into the substantially hollow cylinder when pressed on by a syringe or an IV in order to allow the connection of the syringe or the IV onto the proximal female luer connection.

In another respect, disclosed is an apparatus for protecting an input connection of a medical valve comprising: a substantially hollow cylinder with two or more flaps at a proximal end and an aperture at the distal end; wherein the aperture is configured to receive a portion of the medical valve so that the two or more flaps are positioned above a connection point of the medical valve; and wherein the two or more flaps are configured to fold into the substantially hollow cylinder when pressed on by a syringe or an IV in order to allow the connection of the syringe or the IV onto the connection point of the medical valve.

Numerous additional embodiments are also possible.

### IV. BRIEF DESCRIPTION OF THE DRAWINGS

Other objects and advantages of the invention may become apparent upon reading the detailed description and upon reference to the accompanying drawings.

FIG. 1 is a cross section of a protected medical valve, in accordance with some embodiments.

FIG. 2 is a proximal elevation view of the protected medical valve of FIG. 1, in accordance with some embodiments.

FIG. 3 is a cross section of the protected medical valve of FIG. 1 with the protective members opened to expose the medical valve, in accordance with some embodiments.

FIG. 4 is a proximal elevation view of the protected medical valve of FIG. 3, in accordance with some embodiments.

FIG. 5 is a cross section of the protected medical valve of FIG. 1 with an attached needleless syringe, in accordance with some embodiments.

FIG. 6 is a cross section of a protected medical valve, in accordance with some embodiments.

FIG. 7 is a cross section of the protected medical valve of FIG. 6 with the protective members opened to expose the medical valve, in accordance with some embodiments.

FIG. 8 is a cross section of the protected medical valve of FIG. 6 with an attached syringe, in accordance with some embodiments.

FIG. 9 is a cross section of a protected medical valve, in accordance with some embodiments.

FIG. 10 is a cross section of the protected medical valve of FIG. 9 with the protective members opened to expose the medical valve, in accordance with some embodiments.

FIG. 11 is a cross section of the protected medical valve of FIG. 9 with the protective members opened to expose the medical valve, in accordance with some embodiments.

FIG. 12 is a cross section of the protected medical valve of FIG. 1 with an attached intravenous line, in accordance with some embodiments.

FIG. 13 is a side elevation view of a cover member for a medical valve, in accordance with some embodiments.

FIG. 14 is a side elevation view of the cover member for a medical valve of FIG. 13, in accordance with some embodiments.

FIG. 15 is a proximal elevation view of the cover member of FIG. 14, in accordance with some embodiments.

FIG. 16 is an isometric view of a cover member for a medical valve, in accordance with some embodiments.

FIG. 17 is a proximal elevation view of the cover member of FIG. 16, in accordance with some embodiments.

FIG. 18 is a side elevation view of the cover member of FIG. 16, in accordance with some embodiments.

FIG. 19 is a cross section view taken along the line A-A of FIG. 18 of the cover member of FIG. 16 coupled to a medical valve, in accordance with some embodiments.

FIG. 20 is a photograph of the cover member of FIG. 16 coupled to the medical valve, in accordance with some embodiments.

FIG. 21 is a cross section view taken along the line A-A of FIG. 18 of the cover member of FIG. 16 coupled to the medical valve with an attached needleless syringe, in accordance with some embodiments.

FIG. 22 is a photograph of the cover member of FIG. 16 coupled to the medical valve with the attached needleless syringe, in accordance with some embodiments.

While the invention is subject to various modifications and alternative forms, specific embodiments thereof are shown by way of example in the drawings and the accompanying detailed description. It should be understood, however, that the drawings and detailed description are not intended to limit the invention to the particular embodiments. This disclosure is instead intended to cover all modifications, equivalents, and alternatives falling within the scope of the present invention as defined by the appended claims.

### V. DETAILED DESCRIPTION

One or more embodiments of the invention are described below. It should be noted that these and any other embodiments are exemplary and are intended to be illustrative of the invention rather than limiting. While the invention is widely applicable to different types of systems, it would be impossible or impractical to include all of the possible embodiments and contexts of the invention in this disclosure. Upon reading this disclosure, many alternative embodiments of the present invention will be apparent to persons of ordinary skill in the art.

In the medical field, luer valves and other medical valves are a potential source for the transmission of pathogens into a patient. Currently, the standard of care before inserting a luer lock syringe onto a luer activated valve for intravenous access is to swab the luer activated valve with an alcohol prep pad in order to disinfect the luer activated valve. After swabbing, the luer lock syringe may be coupled to the luer activated valve to dispense the medication or solution into the patient. When the syringe is not coupled to the valve, the valve is generally just left exposed to the elements and thus the standard of care is to first use the alcohol prep pad.

Caps and pouches have been developed to protect the valve from contamination. Lawrence A. Lynn disclosed in U.S. Pat. No. 8,480,968 a disinfectant swab-pouch which would snuggly receive and elastically retain a luer valve when the luer valve was not in use. The issue with this pouch or any similar cap is that the user must first remove the cap or pouch before a syringe may be coupled to the valve. When the pouch or cap is removed it may become lost and/or contaminated and thus have to be replaced.

A need exists for an apparatus for protecting a medical valve from contamination while permitting the simple and quick access to the valve with a syringe. The embodiment or embodiments described herein solve these problems and others by proposing a new protected medical valve which allows for simple and quick valve access with both needle and needleless syringes.

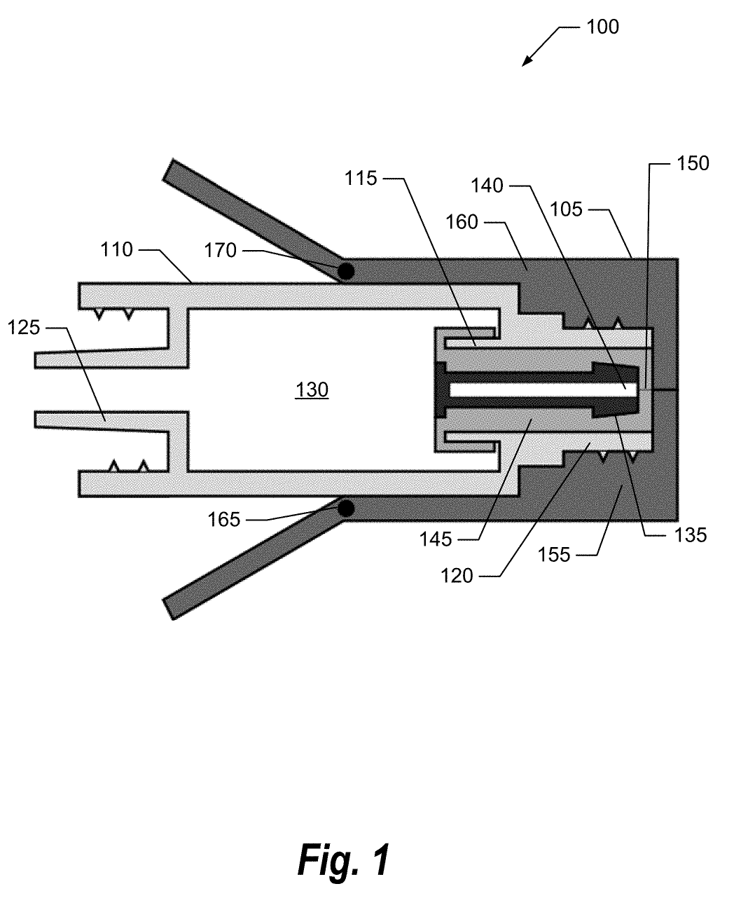

FIG. 1 is a cross section of a protected medical valve, in accordance with some embodiments.

In some embodiments, the apparatus 100 comprises a cover member 105 and a luer activated valve 110. The luer activated valve 110 comprises a valve 115 within a proximal female luer connection 120, a distal male luer tip 125, and a fluid passageway 130. The fluid may either be in the liquid or gas state. The valve comprises a rigid plastic piston 135, having a central fluid passageway 140 at the proximal end and one or more radial fluid passageway openings towards the distal end of the rigid plastic piston 135, surrounded by a flexible elastomeric material 145 having a proximal slit 150 which permits fluid passageway into the central fluid passageway 140. The cover member 105 comprises one or more sections which together cover and protect the proximal female luer connection from contamination. In the embodiment illustrated in FIG. 1, the cover member 105 comprises two sections 155 and 160 which are hinged or pivoted with spring hinges 165 and 170, respectively. The cover member sections may fit snugly around the female luer connection as illustrated, but in some other embodiments the cover member sections may not be in contact with the luer connection. In some embodiments, the surfaces of the cover members, especially the inner surfaces facing the valve, may comprise an antibacterial coating material such as silver or silver salts such as sulfadiazine. The dimensions and properties of the luer connection and tip conform to the ISO 80369 standard and may comprise a luer-lock or a luer-slip.

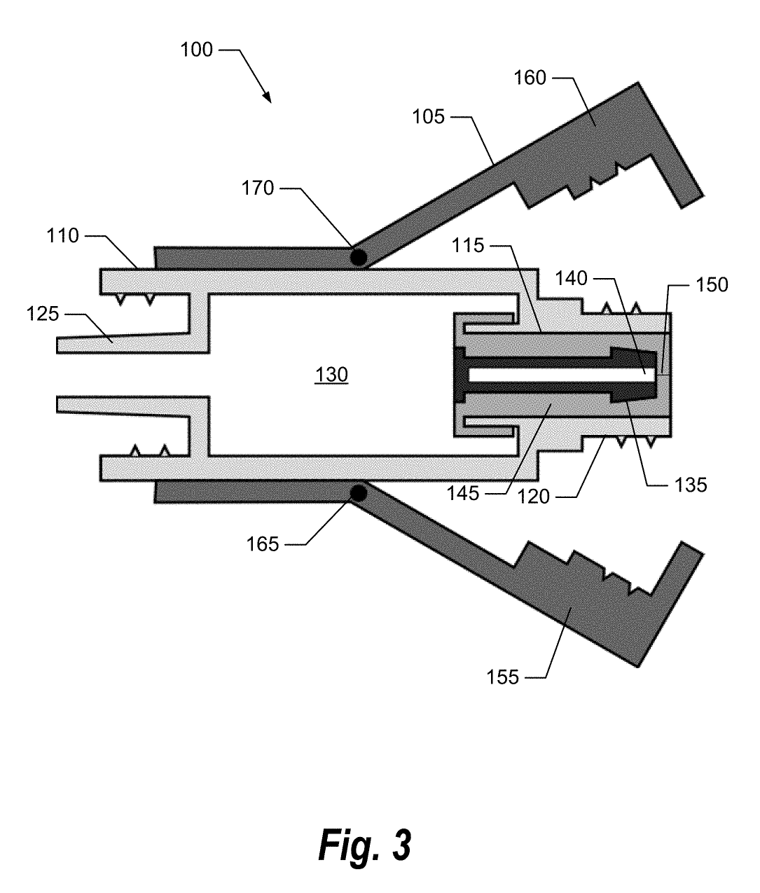

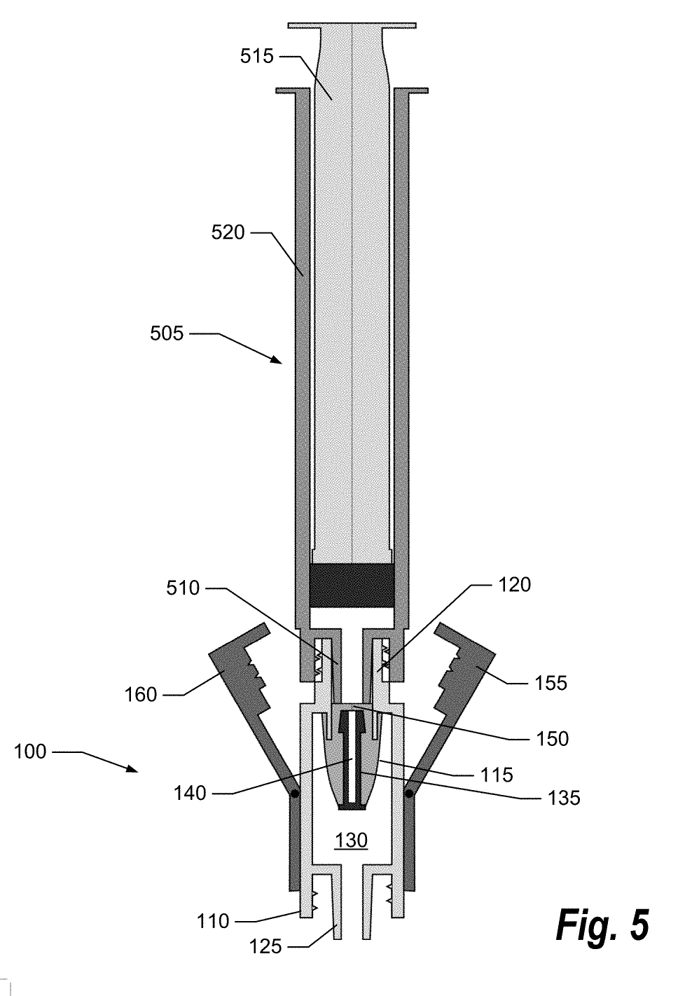

When the apparatus is to be used, it is removed from its sterile package and the distal male luer tip is coupled to an intravenous line (IV), vial adapter, or any other valve where the coupling point is not protected from contamination. To ensure sterility of the connection, the coupling point of the IV line, vial adapter, or valve is disinfected prior to making the connection. When fluids are to be introduced into the fluid passageway, the lever arms of the cover member sections are pressed toward the housing of the luer activated valve to expose the proximal female luer connection as illustrated in FIG. 3 and a luer lock syringe is coupled to the female luer connection of the apparatus as illustrated in FIG. 5. After the fluids have been dispensed from the syringe, the syringe may be removed from the female luer connection so that the spring hinges may automatically close the cover members over the valve in order to protect the valve from contamination.

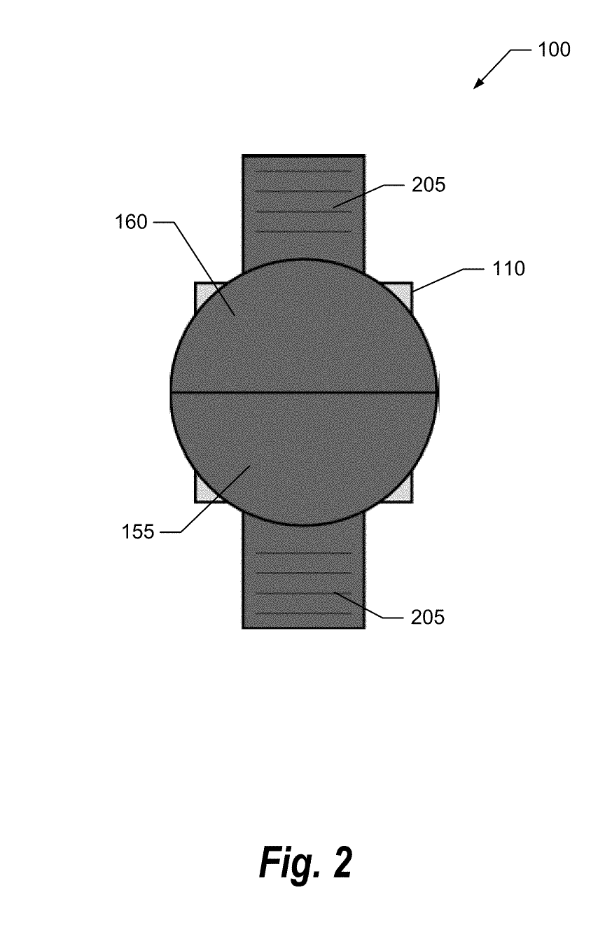

FIG. 2 is a proximal elevation view of the protected medical valve of FIG. 1, in accordance with some embodiments.

In some embodiments, when the apparatus 100 is in the closed position, the cover member sections 155 and 160 make contact with one another to protect the female luer connection from contamination. The levers of the cover member sections may have one or more ridges 205 to make it easier for a user to press down on the levers. The housing of the luer activated valve is visible in the proximal elevation view.

FIG. 3 is a cross section of the protected medical valve of FIG. 1 with the protective members opened to expose the medical valve, in accordance with some embodiments.

In some embodiments, the apparatus 100 comprises a cover member 105 and a luer activated valve 110. The luer activated valve 110 comprises a valve 115 within a proximal female luer connection 120, a distal male luer tip 125, and a fluid passageway 130. The valve comprises a rigid plastic piston 135, having a central fluid passageway 140 at the proximal end and one or more radial fluid passageway openings towards the distal end of the rigid plastic piston 135, surrounded by a flexible elastomeric material 145 having a proximal slit 150 which permits fluid passageway into the central fluid passageway 140. The cover member 105 comprises one or more sections which together cover and protect the proximal female luer connection from contamination. In the embodiment illustrated in FIG. 3, the cover member 105 comprises two sections 155 and 160 which are hinged or pivoted with spring hinges 165 and 170, respectively. When the spring hinges are compressed by pressing down on the lever arms, the cover member sections separate to expose the female luer connection as illustrated. In some embodiments, the surfaces of the cover member, especially the inner surfaces facing the valve, may comprise an antibacterial coating material such as silver or silver salts such as sulfadiazine. The dimensions and properties of the luer connection and tip conform to the ISO 80369 standard and may comprise a luer-lock or a luer-slip.

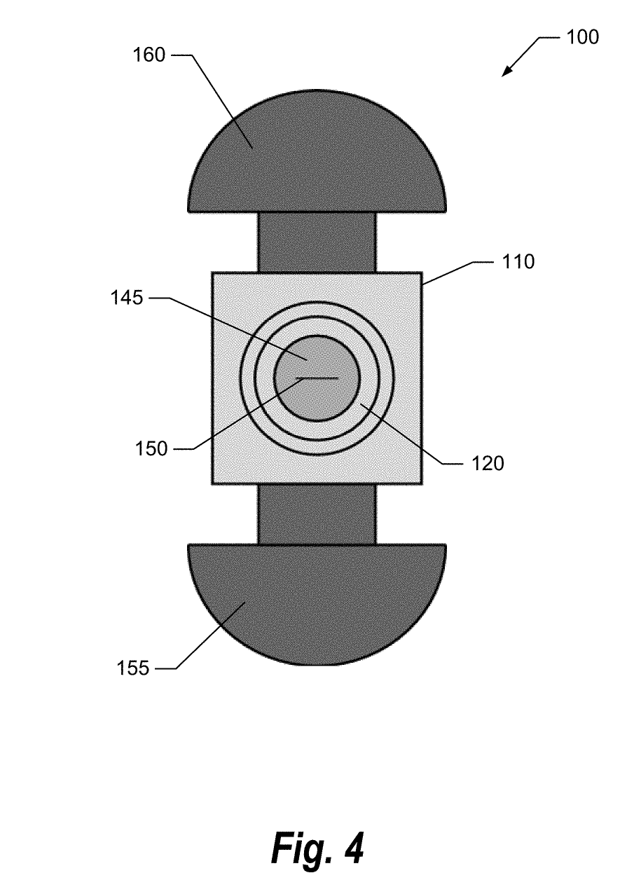

FIG. 4 is a proximal elevation view of the protected medical valve of FIG. 3, in accordance with some embodiments.

In some embodiments, when the apparatus 100 is in the open position, the cover member sections 155 and 160 are separated from one another to expose the proximal female luer connection 120 of the luer activated valve 110. In the view illustrated in FIG. 4, the flexible elastomeric material 145 and the proximal slit 150 of the valve are visible.

FIG. 5 is a cross section of the protected medical valve of FIG. 1 with an attached needleless syringe, in accordance with some embodiments.

In some embodiments, when the apparatus 100 is in the open position, the cover member sections 155 and 160 are separated from one another and the proximal female luer connection 120 of the luer activated valve 110 is exposed so that a syringe 505 may be coupled to the valve. When the syringe is threaded onto the outside threads of the female luer connection 120, the tip 510 of the syringe presses the valve 115 into the housing of the luer activated valve 110. Pressing the plunger 515 into the barrel 520 of the syringe causes the fluid to pass through the tip 510 of the syringe, through the proximal slit 150, through the central fluid passageway 140 of the rigid plastic piston 135, through the one or more radial fluid passageway openings, and into the fluid passageway 130 of the luer activated valve 110 and eventually through the distal male luer tip 125 and into an IV line or other valve.

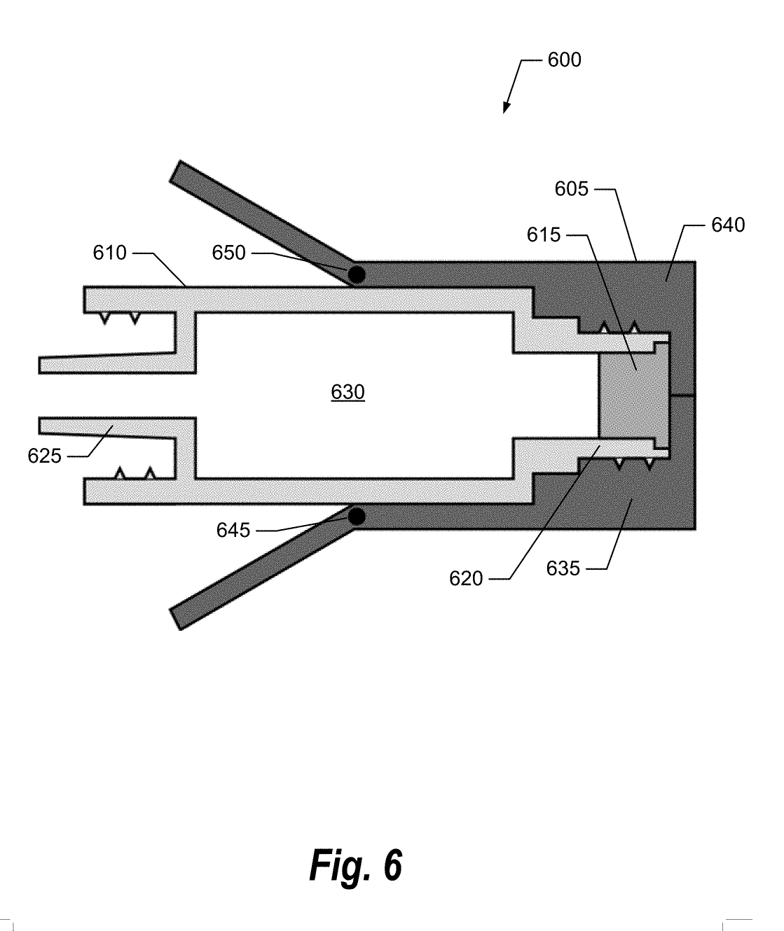

FIG. 6 is a cross section of a protected medical valve, in accordance with some embodiments.

In some embodiments, the apparatus 600 comprises a cover member 605, a housing 610, and a septum 615. The housing 610 comprises a proximal female luer connection 620, a distal male luer tip 625, and a fluid passageway 630. The cover member 605 comprises one or more sections which together cover and protect the proximal female luer connection from contamination. In the embodiment illustrated in FIG. 6, the cover member 605 comprises two sections 635 and 640 which are hinged or pivoted with spring hinges 645 and 650, respectively. The cover member sections may fit snugly around the female luer connection as illustrated, but in some other embodiments the cover member sections may not be in contact with the luer connection. In some embodiments, the surfaces of the cover members, especially the inner surfaces facing the proximal female luer connection, may comprise an antibacterial coating material such as silver or silver salts such as sulfadiazine. The dimensions and properties of the luer connection and tip conform to the ISO 80369 standard and may comprise a luer-lock or a luer-slip.

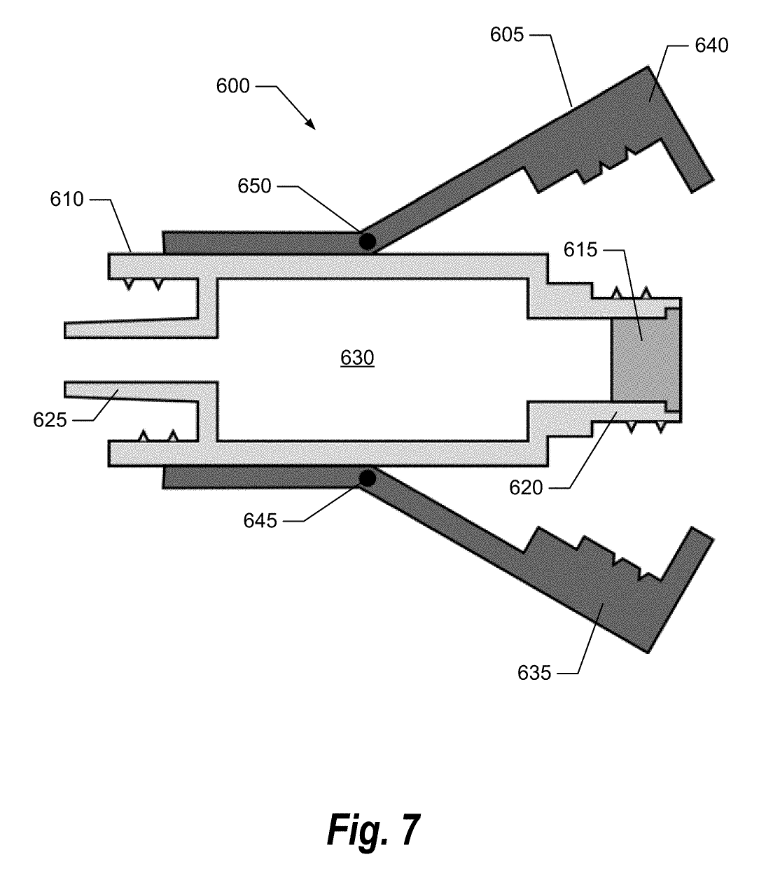

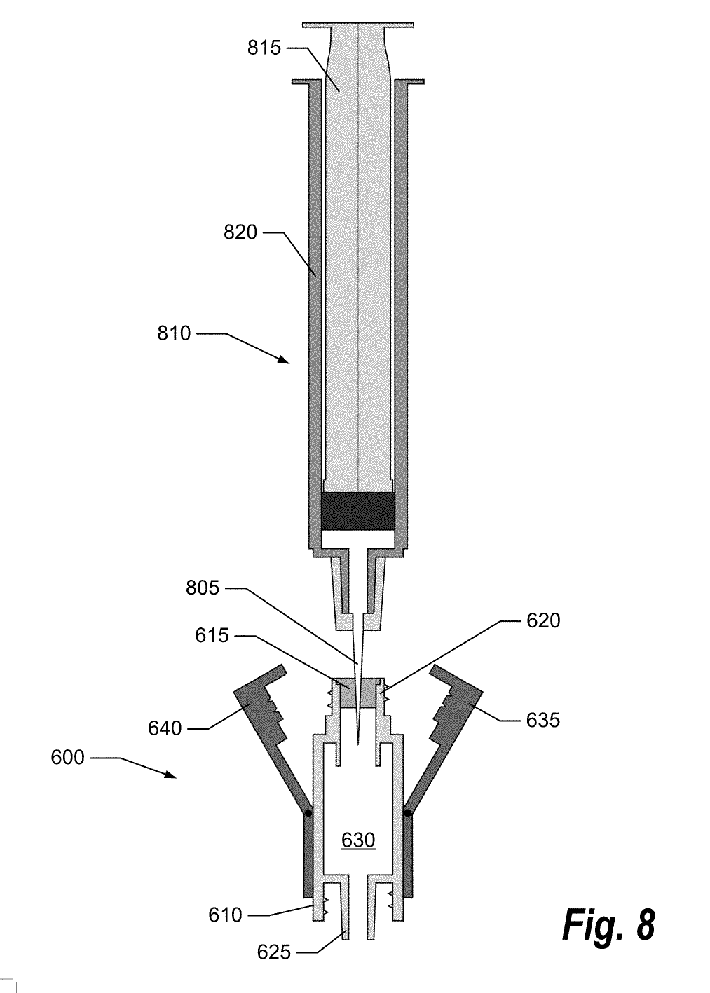

When the apparatus is to be used, it is removed from its sterile package and the distal male luer tip is coupled to an IV, vial adapter, or any other valve where the coupling point is not protected from contamination. To ensure sterility of the connection, the coupling point of the IV line, vial adapter, or valve is disinfected prior to making a connection. When fluids are to be introduced into the fluid passageway, the lever arms of the cover member sections are pressed toward the housing to expose the proximal female luer connection as illustrated in FIG. 7 and a syringe having a needle is inserted through the septum of the apparatus as illustrated in FIG. 8. After the fluids have been dispensed from or drawn into the syringe, the syringe may be removed from the female luer connection so that the spring hinges may automatically close the cover members over the proximal female luer connection in order to protect the proximal female luer connection from contamination.

FIG. 7 is a cross section of the protected medical valve of FIG. 6 with the protective members opened to expose the medical valve, in accordance with some embodiments.

In some embodiments, the apparatus 600 comprises a cover member 605, a housing 610, and a septum 615. The housing 610 comprises a proximal female luer connection 620, a distal male luer tip 625, and a fluid passageway 630. The cover member 605 comprises one or more sections which together cover and protect the proximal female luer connection from contamination. In the embodiment illustrated in FIG. 7, the cover member 605 comprises two sections 635 and 640 which are hinged or pivoted with spring hinges 645 and 650, respectively. When the spring hinges are compressed by pressing down on the lever arms, the cover member sections separate to expose the female luer connection as illustrated. In some embodiments, the surfaces of the cover member, especially the inner surfaces facing the female luer connection, may comprise an antibacterial coating material such as silver or silver salts such as sulfadiazine. The dimensions and properties of the luer connection and tip conform to the ISO 80369 standard and may comprise a luer-lock or a luer-slip.

FIG. 8 is a cross section of the protected medical valve of FIG. 6 with an attached syringe, in accordance with some embodiments.

In some embodiments, when the apparatus 600 is in the open position, the cover member sections 635 and 640 are separated from one another and the proximal female luer connection 620 of the housing 610 is exposed so that a needle 805 of syringe 810 may be inserted through the septum 615 of the apparatus. Once the needle 805 is inserted through the septum 615, the plunger 815 may be pressed into the barrel 820 of the syringe 810 to transfer the fluid of the syringe into the fluid passageway 630 of the apparatus 600 and eventually through the distal male luer tip 625 and into an IV line or other valve.

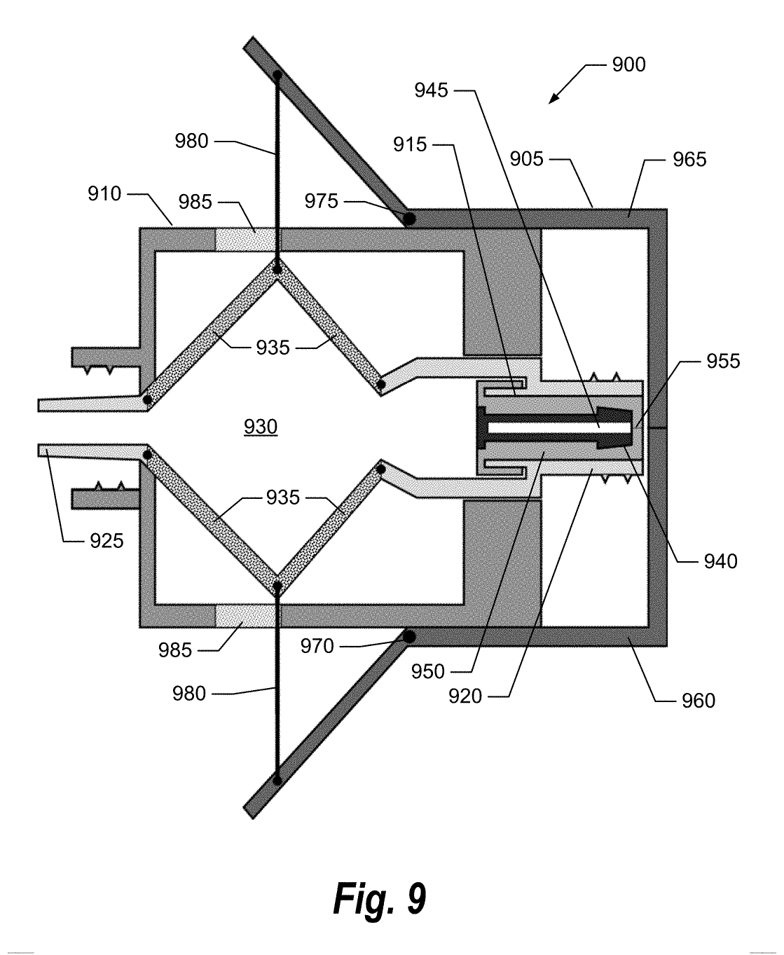

FIG. 9 is a cross section of a protected medical valve, in accordance with some embodiments.

In some embodiments, the apparatus 900 comprises a cover member 905 and a luer activated valve 910. The luer activated valve 910 comprises a valve 915 within a proximal female luer connection 920, a distal male luer tip 925, a fluid passageway 930, and an extending bellow 935. The fluid may either be in the liquid or gas state. The valve comprises a rigid plastic piston 940, having a central fluid passageway 945 at the proximal end and one or more radial fluid passageway openings towards the distal end of the rigid plastic piston 940, surrounded by a flexible elastomeric material 950 having a proximal slit 955 which permits fluid passageway into the central fluid passageway 945. The cover member 905 comprises one or more sections which together cover and protect the proximal female luer connection from contamination. In the embodiment illustrated in FIG. 9, the cover member 905 comprises two sections 960 and 965 which are hinged or pivoted with spring hinges 970 and 975, respectively. Connecting rods 980 are coupled between the levers of the cover member sections 960 and 965 and the extending bellow 935. The connecting rods 980 pass through an opening 985 in the side of the luer activated valve 910. The cover member sections may not be in contact with the female luer connection as illustrated, but in some other embodiments the cover member sections may fit snugly around the luer connection. In some embodiments, the surfaces of the cover members, especially the inner surfaces facing the valve, may comprise an antibacterial coating material such as silver or silver salts such as sulfadiazine. The dimensions and properties of the luer connection and tip conform to the ISO 80369 standard and may comprise a luer-lock or a luer-slip. In some embodiments, the luer activated valve comprises a housing and a septum as illustrated and described in FIG. 6, FIG. 7, and FIG. 8.

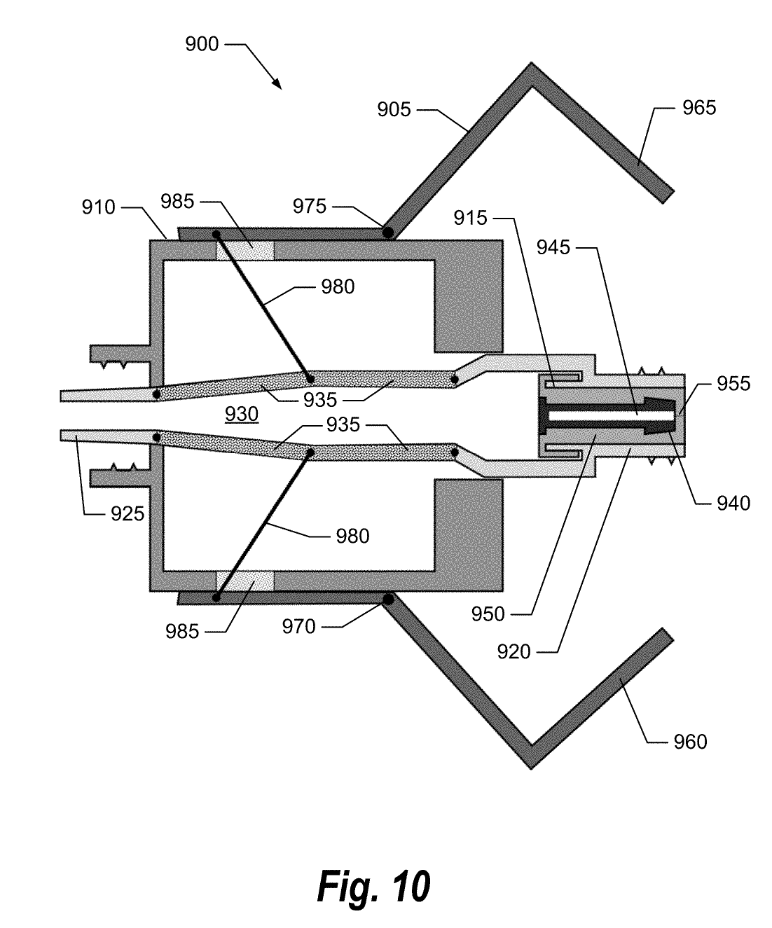

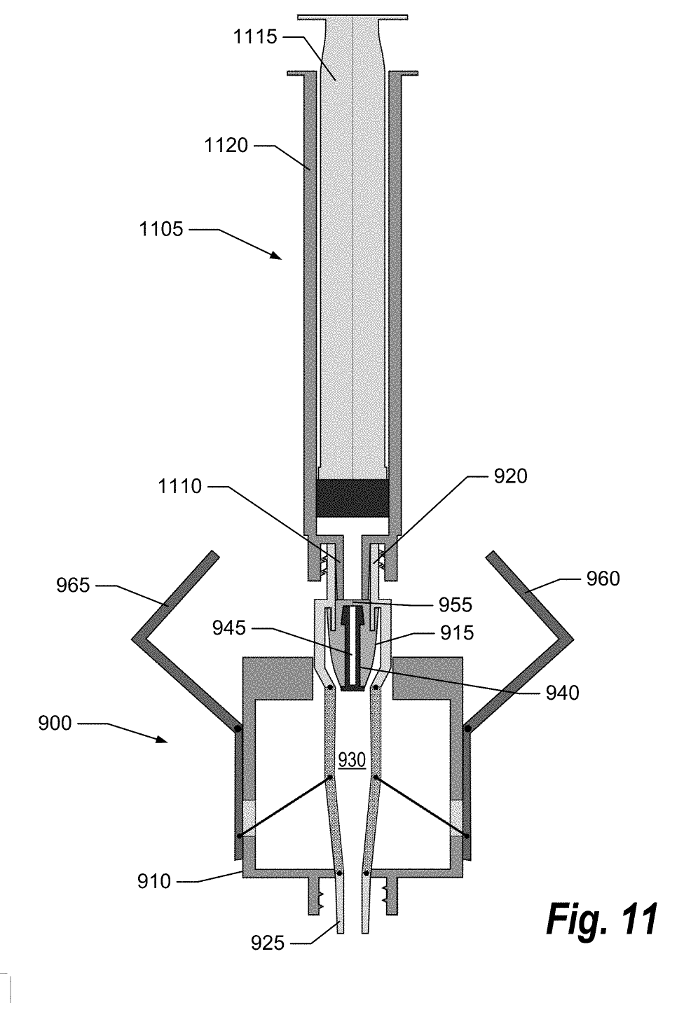

When the apparatus is to be used, it is removed from its sterile package and the distal male luer tip is coupled to an IV, vial adapter, or any other valve where the coupling point is not protected from contamination. To ensure sterility of the connection, the coupling point of the IV line, vial adapter, or valve is disinfected prior to making the connection. When fluids are to be introduced into the fluid passageway, the lever arms of the cover member sections are pressed toward the housing of the luer activated valve which causes the bellow to extend to protrude and expose the proximal female luer connection as illustrated in FIG. 10 and a luer lock syringe is coupled to the female luer connection of the apparatus as illustrated in FIG. 11. After the fluids have been dispensed from the syringe, the syringe may be removed from the female luer connection so that the spring hinges may automatically close the cover members over the valve in order to protect the valve from contamination.

FIG. 10 is a cross section of the protected medical valve of FIG. 9 with the protective members opened to expose the medical valve, in accordance with some embodiments.

In some embodiments, the apparatus 900 comprises a cover member 905 and a luer activated valve 910. The luer activated valve 910 comprises a valve 915 within a proximal female luer connection 920, a distal male luer tip 925, a fluid passageway 930, and an extending bellow 935. The valve comprises a rigid plastic piston 940, having a central fluid passageway 945 at the proximal end and one or more radial fluid passageway openings towards the distal end of the rigid plastic piston 945, surrounded by a flexible elastomeric material 950 having a proximal slit 955 which permits fluid passageway into the central fluid passageway 945. The cover member 905 comprises one or more sections which together cover and protect the proximal female luer connection from contamination. In the embodiment illustrated in FIG. 10, the cover member 905 comprises two sections 960 and 965 which are hinged with spring hinges 970 and 975, respectively. Connecting rods 980 are coupled between the levers of the cover member sections 960 and 965 and the extending bellow 935. The connecting rods 980 pass through an opening 985 in the side of the luer activated valve 910. When the spring hinges are compressed by pressing down on the lever arms of the cover member sections, the cover member sections separate and the bellow is compressed to expose and protrude the female luer connection as illustrated. The protrusion of the female luer connection facilitates the attachment of the apparatus to a syringe. In some embodiments, the surfaces of the cover member, especially the inner surfaces facing the valve, may comprise an antibacterial coating material such as silver or silver salts such as sulfadiazine. The dimensions and properties of the luer connection and tip conform to the ISO 80369 standard and may comprise a luer-lock or a luer-slip.

FIG. 11 is a cross section of the protected medical valve of FIG. 9 with the protective members opened to expose the medical valve, in accordance with some embodiments.

In some embodiments, when the apparatus 900 is in the open position, the cover member sections 960 and 965 are separated from one another and the proximal female luer connection 920 of the luer activated valve 910 is exposed so that a syringe 1105 may be coupled to the valve. When the syringe is threaded onto the outside threads of the female luer connection 920, the tip 1110 of the syringe presses the valve 915 into the housing of the luer activated valve 910. Pressing the plunger 1115 into the barrel 1120 of the syringe causes the fluid to pass through the tip 1110 of the syringe, through the proximal slit 955, through the central fluid passageway 945 of the rigid plastic piston 940, through the one or more radial fluid passageway openings, and into the fluid passageway 930 of the luer activated valve 910 and eventually through the distal male luer tip 925 and into an IV line or other valve.

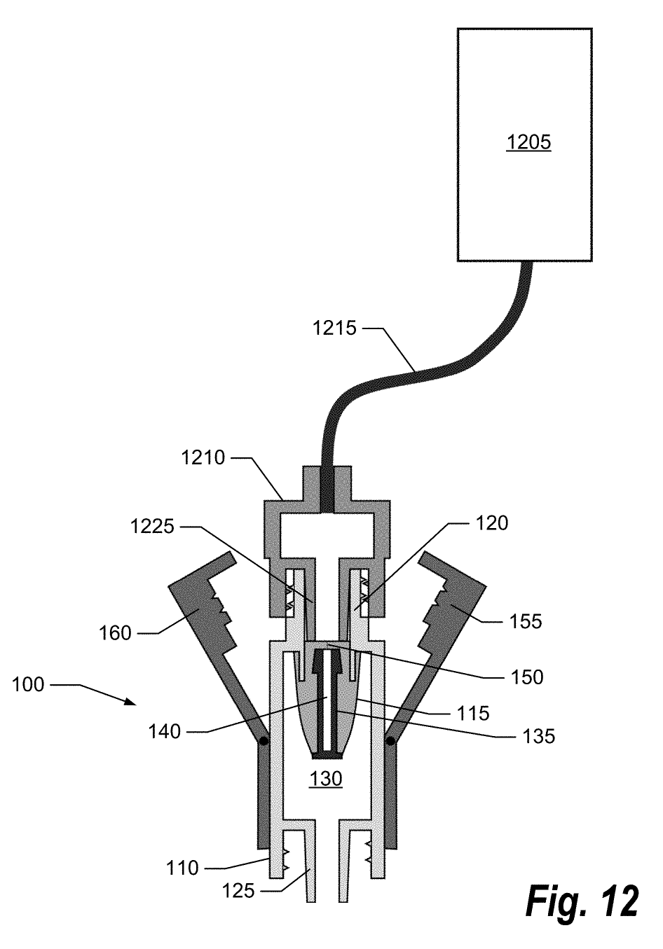

FIG. 12 is a cross section of the protected medical valve of FIG. 1 with an attached intravenous line, in accordance with some embodiments.

In some embodiments, when the apparatus 100 is in the open position, the cover member sections 155 and 160 are separated from one another and the proximal female luer connection 120 of the luer activated valve 110 is exposed so that an IV 1205 containing IV fluids may be coupled to the valve. The IV is coupled to the apparatus with a male luer adapter 1210 through tubing 1215. When the male luer adapter 1210 is threaded onto the outside threads of the female luer connection 120, the tip 1225 of the male luer adapter presses the valve 115 into the housing of the luer activated valve 110. Gravitational forces cause the IV fluid to pass through the tubing 1215, through the male luer adapter tip 1225, through the proximal slit 150, through the central fluid passageway 140 of the rigid plastic piston 135, through the one or more radial fluid passageway openings, and into the fluid passageway 130 of the luer activated valve 110 and eventually through the distal male luer tip 125 and into an IV line or other valve. The apparatuses 600 and 900 illustrated in FIG. 6, FIG. 7, FIG. 9, and FIG. 10 may also be similarly coupled to an IV 1205.

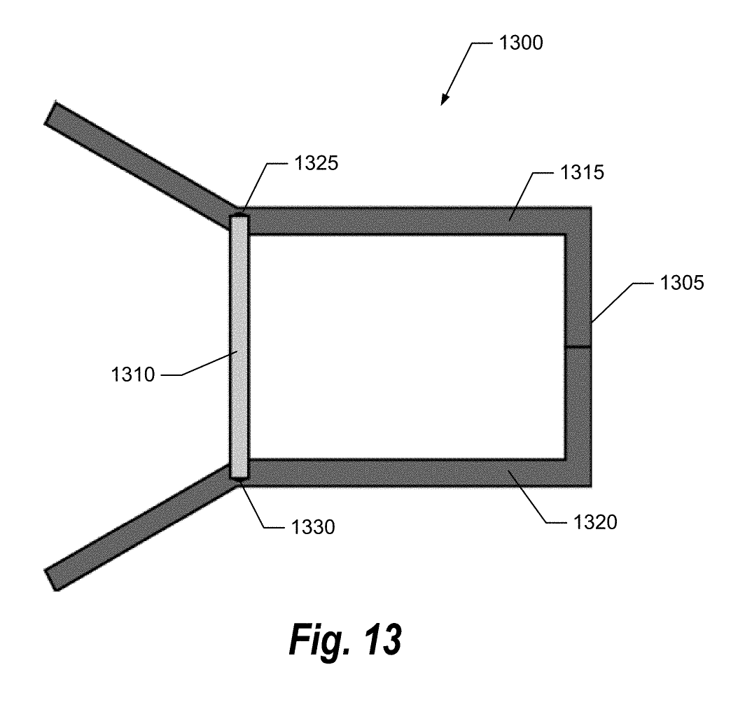

FIG. 13 is a side elevation view of a cover member for a medical valve, in accordance with some embodiments.

In some embodiments, apparatus 1300 comprises a cover member 1305 and a coupling member 1310. In the embodiment illustrated in FIG. 13, the cover member comprises two cover member sections 1315 and 1320 each individually pivoted with spring hinges 1325 and 1330, respectively. The coupling member 1310 may comprise an elastic material which may easily be slipped onto and coupled to any unprotected medical valve. Once the apparatus is attached to an unprotected medical valve, the cover member will protect the input connection of the medical valve from contamination, similar to the embodiments illustrated in FIG. 1, FIG. 6, and FIG. 9. In some embodiments, the surfaces of the cover members, especially the inner surfaces facing the valve, may comprise an antibacterial coating material such as silver or silver salts such as sulfadiazine.

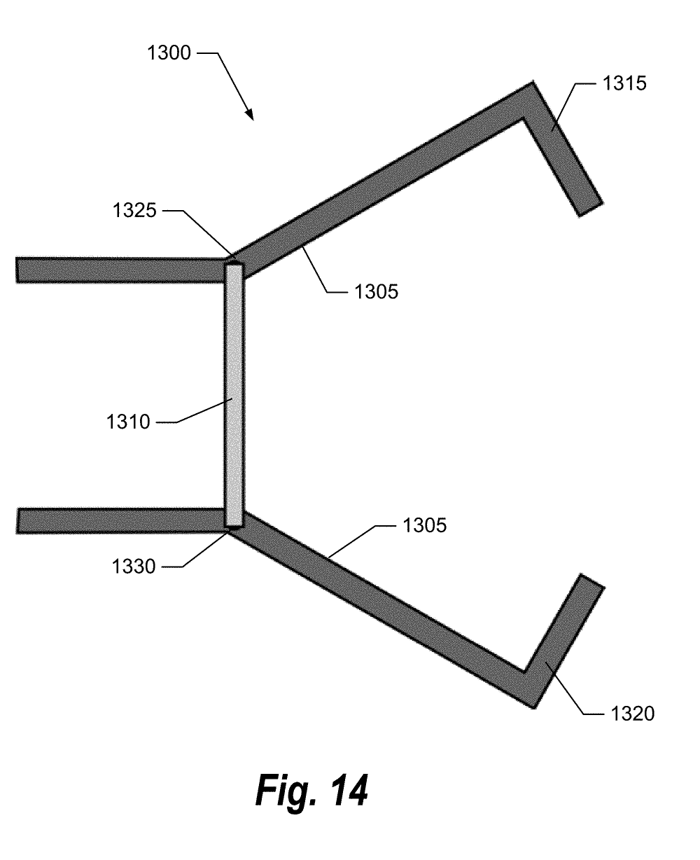

FIG. 14 is a side elevation view of the cover member for a medical valve of FIG. 13, in accordance with some embodiments.

In some embodiments, apparatus 1300 comprises a cover member 1305 and a coupling member 1310. In the embodiment illustrated in FIG. 13, the cover member comprises two cover member sections 1315 and 1320 each individually pivoted with spring hinges 1325 and 1330, respectively. The coupling member 1310 may comprise an elastic material which may easily be slipped onto and coupled to any unprotected medical valve. Once the apparatus is attached to an unprotected medical valve, the cover member may be opened, while still remaining coupled to the medical valve, to expose the input connection of the medical valve for connection to a syringe or IV, similar to the embodiments illustrated in FIG. 3, FIG. 4, FIG. 5, FIG. 7, FIG. 8, FIG. 10, FIG. 11, and FIG. 12. In some embodiments, the surfaces of the cover members, especially the inner surfaces facing the valve, may comprise an antibacterial coating material such as silver or silver salts such as sulfadiazine.

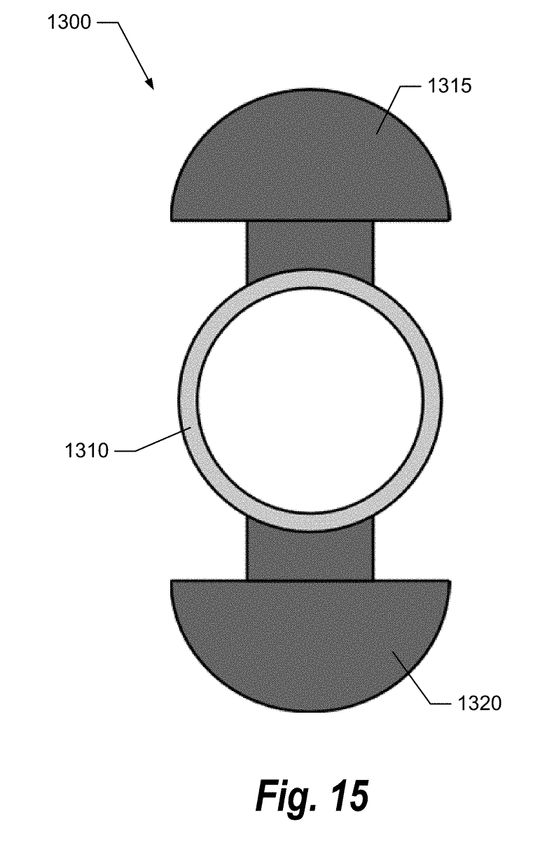

FIG. 15 is a proximal elevation view of the cover member of FIG. 14, in accordance with some embodiments.

In some embodiments, when the apparatus 1300 is in the open position, the cover member sections 1315 and 1320 are separated from one another by pivoting around the spring hinges in order to expose the input connection of the medical valve, such as a female luer connection. In the view illustrated in FIG. 15, the coupling member 1310 is visible. The coupling member is configured to snugly fit around an unprotected medical valve to protect the medical valve from contamination.

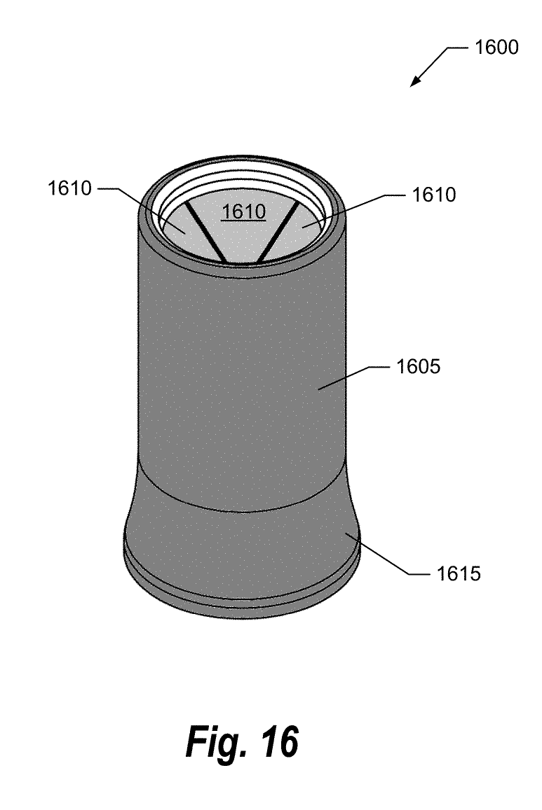

FIG. 16 is an isometric view of a cover member for a medical valve, in accordance with some embodiments.

In some embodiments, apparatus 1600 comprises a cover member 1605 comprising a substantially hollow cylinder having four flaps 1610 at the proximal end and an aperture at the distal end. The cover member 1605 may comprise an elastic material which may easily be slipped onto and coupled to any unprotected medical valve. In some embodiments, the cover member 1605 has a flared out outer wall 1615 to facilitate the placement of the cover member onto an unprotected medical valve. Once the apparatus is attached to an unprotected medical valve, the cover member will protect the input connection of the medical valve from contamination, similar to the embodiments illustrated in FIG. 1, FIG. 6, and FIG. 9. In some embodiments, the surfaces of the cover member, especially the inner surfaces facing the medical valve, may comprise an antibacterial coating material such as silver or silver salts such as sulfadiazine.

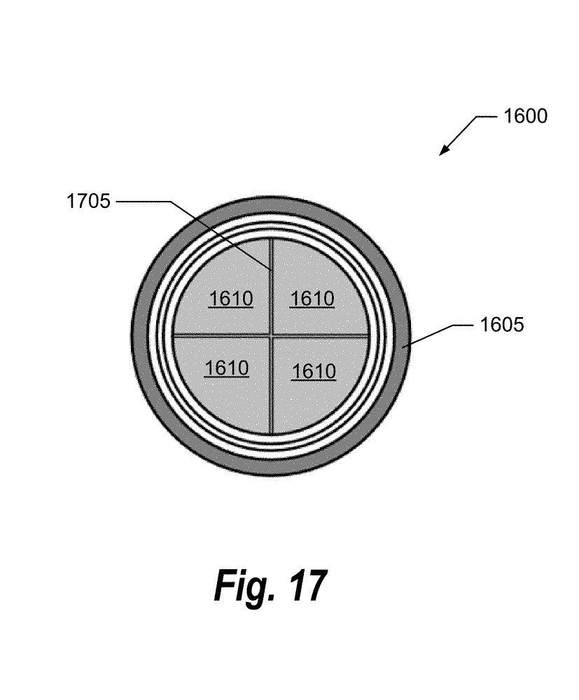

FIG. 17 is a proximal elevation view of the cover member of FIG. 16, in accordance with some embodiments.

In some embodiments, apparatus 1600 comprises a cover member 1605 having four flaps 1610 at the proximal end and an aperture at the distal end. When pressed, the four flaps 1610 will bend into the interior void of the cover member to expose the input portion of a medical valve, such as a proximal female luer connection. A syringe or an IV may then be coupled to the exposed input portion of the medical valve. In other embodiments, the cover member may comprise two, three, or five or more flaps.

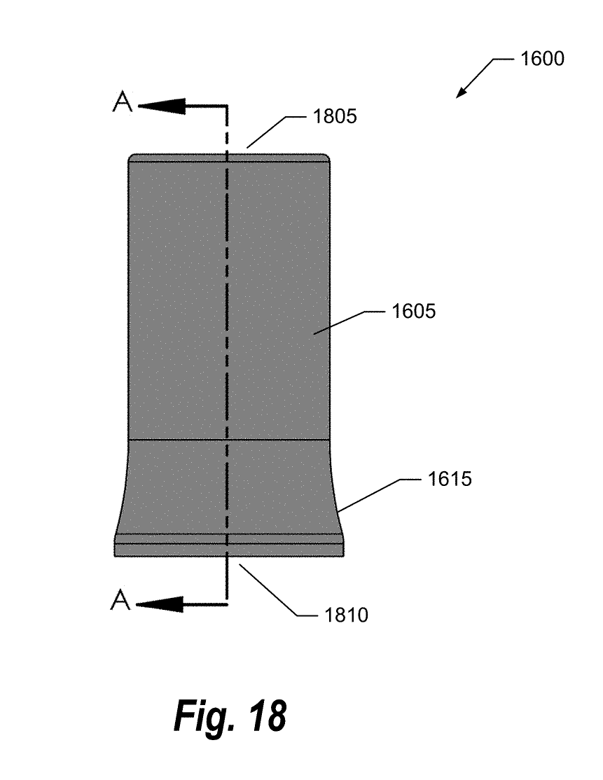

FIG. 18 is a side elevation view of the cover member of FIG. 16, in accordance with some embodiments.

In some embodiments, apparatus 1600 comprises a cover member 1605 having four flaps at the proximal end 1805 and an aperture at the distal end 1810. The cover member 1605 may comprise an elastic material which may easily be slipped onto and coupled to any unprotected medical valve. In some embodiments, the cover member 1605 has a flared out outer wall 1615 to facilitate the placement of the cover member onto an unprotected medical valve.

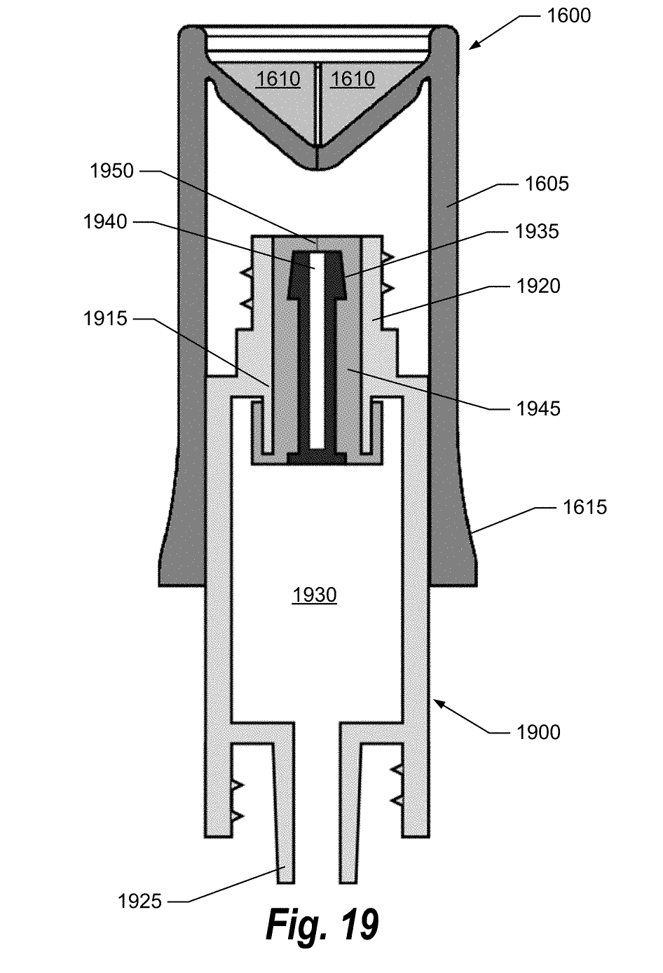

FIG. 19 is a cross section view taken along the line A-A of FIG. 18 of the cover member of FIG. 16 coupled to a medical valve, in accordance with some embodiments.

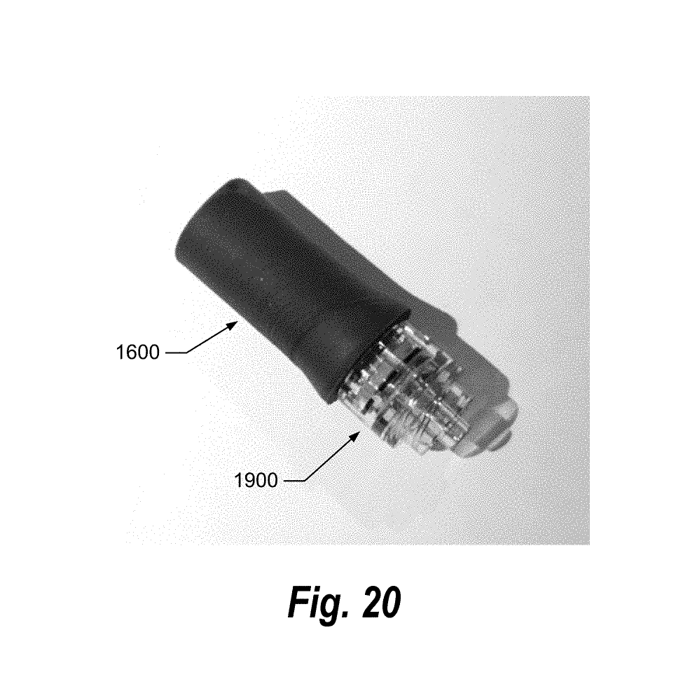

FIG. 20 is a photograph of the cover member of FIG. 16 coupled to the medical valve, in accordance with some embodiments.

In some embodiments, apparatus 1600 comprises a cover member 1605 having four flaps 1610 at the proximal end and an aperture at the distal end. The cover member 1605 may comprise an elastic material which may easily be slipped onto and coupled to any unprotected medical valve 1900. In some embodiments, the cover member 1605 has a flared out outer wall 1615 to facilitate the placement of the cover member onto an unprotected medical valve 1900. The unprotected medical valve 1900, illustrated in FIG. 19 as a luer activated valve, comprises a valve 1915 within a proximal female luer connection 1920, a distal male luer tip 1925, and a fluid passageway 1930. The fluid may either be in the liquid or gas state. The valve comprises a rigid plastic piston 1935, having a central fluid passageway 1940 at the proximal end and one or more radial fluid passageway openings towards the distal end of the rigid plastic piston 1935, surrounded by a flexible elastomeric material 1945 having a proximal slit 1950 which permits fluid passageway into the central fluid passageway 1930. Once the apparatus 1600 is attached to the unprotected medical valve 1900, as illustrated in FIG. 20, the cover member 1605 along with the flaps 1610 will protect the proximal female luer connection 1920 input connection of the medical valve 1900 from contamination, similar to the embodiments illustrated in FIG. 1, FIG. 6, and FIG. 9.

In some embodiments, the apparatus 1600 and medical valve 1900 are integrated into a single device. In such an embodiment, the user is not capable of removing the apparatus 1600 from the medical valve 1900.

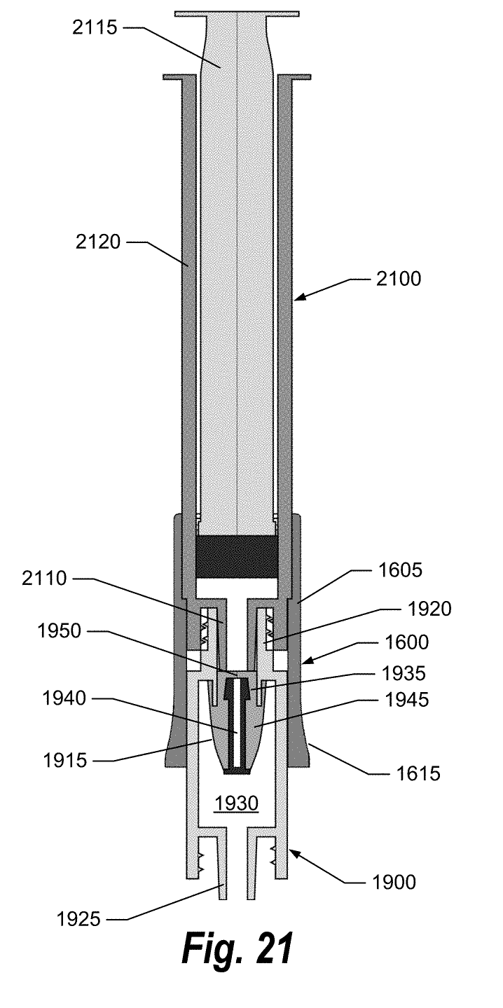

FIG. 21 is a cross section view taken along the line A-A of FIG. 18 of the cover member of FIG. 16 coupled to the medical valve with an attached needleless syringe, in accordance with some embodiments.

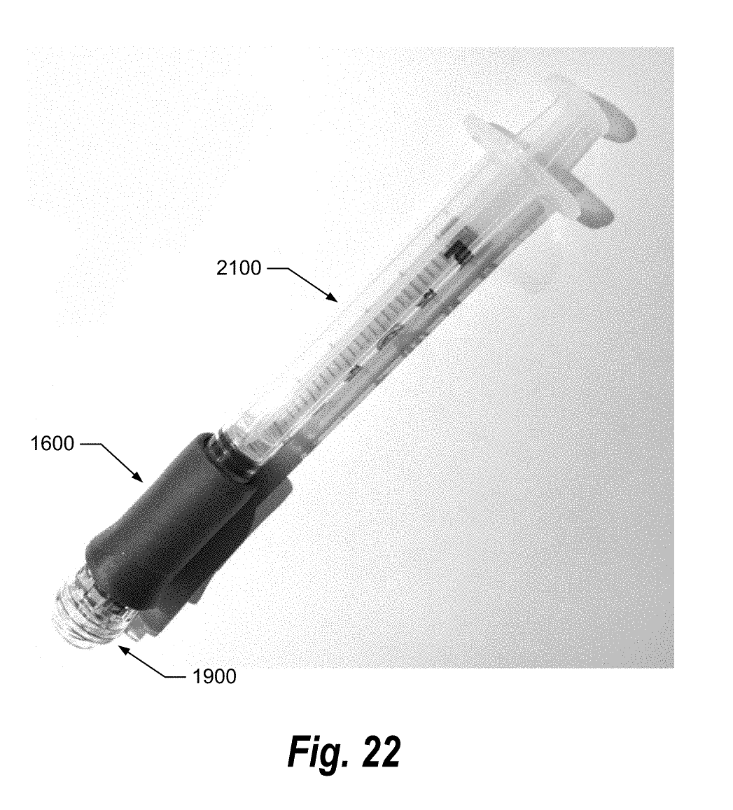

FIG. 22 is a photograph of the cover member of FIG. 16 coupled to the medical valve with the attached needleless syringe, in accordance with some embodiments.

In some embodiments, apparatus 1600 comprises a cover member 1605 having four flaps at the proximal end and an aperture at the distal end. The cover member 1605 may comprise an elastic material made from polypropylene, polycarbonate, nylon, PVDF, or other similar material, which may permit the cover member to be easily slipped onto and coupled to any unprotected medical valve 1900. In some embodiments, the cover member 1605 has a flared out outer wall 1615 to facilitate the placement of the cover member onto the unprotected medical valve 1900. The unprotected medical valve 1900, illustrated in FIG. 21 as a luer activated valve, comprises a valve 1915 within a proximal female luer connection 1920, a distal male luer tip 1925, and a fluid passageway 1930. The fluid may either be in the liquid or gas state. The valve comprises a rigid plastic piston 1935, having a central fluid passageway 1940 at the proximal end and one or more radial fluid passageway openings towards the distal end of the rigid plastic piston 1935, surrounded by a flexible elastomeric material 1945 having a proximal slit 1950 which permits fluid passageway into the central fluid passageway 1940. Once the apparatus 1600 is attached to the unprotected medical valve 1900, as illustrated in FIG. 21, the cover member 1605 along with the flaps will protect the proximal female luer connection 1920 input connection of the medical valve 1900 from contamination, similar to the embodiments illustrated in FIG. 1, FIG. 6, FIG. 9, and FIG. 19. When a syringe 2100 is threaded onto the outside threads of the female luer connection 1920, by first pushing the tip 2110 of the syringe through flaps of the apparatus 1600, the tip 2110 of the syringe presses the valve 1915 into the housing of the luer activated valve 1900. Pressing the plunger 2115 into the barrel 2120 of the syringe causes the fluid to pass through the tip 2110 of the syringe, through the proximal slit 1950, through the central fluid passageway 1940 of the rigid plastic piston 1935, through the one or more radial fluid passageway openings, and into the fluid passageway 1930 of the luer activated valve 1900 and eventually through the distal male luer tip 1925 and into an IV line or other valve.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

The benefits and advantages that may be provided by the present invention have been described above with regard to specific embodiments. These benefits and advantages, and any elements or limitations that may cause them to occur or to become more pronounced are not to be construed as critical, required, or essential features of any or all of the claims. As used herein, the terms “comprises,” “comprising,” or any other variations thereof, are intended to be interpreted as non-exclusively including the elements or limitations which follow those terms. Accordingly, a system, method, or other embodiment that comprises a set of elements is not limited to only those elements, and may include other elements not expressly listed or inherent to the claimed embodiment.

While the present invention has been described with reference to particular embodiments, it should be understood that the embodiments are illustrative and that the scope of the invention is not limited to these embodiments. Many variations, modifications, additions, and improvements to the embodiments described above are possible. It is contemplated that these variations, modifications, additions, and improvements fall within the scope of the invention as detailed within the following claims.

1. An apparatus comprising:
a housing having a proximal female luer connection, a valve within the proximal female luer connection, a distal male luer tip, and a fluid passageway between the proximal female luer connection and the distal male luer tip; and
a cover member comprising a substantially hollow cylinder with two or more flaps at a proximal end and an aperture at the distal end;
wherein the aperture of the cover member is configured to receive the housing so that the two or more flaps are positioned above the proximal female luer connection; and
wherein the two or more flaps are configured to fold into the substantially hollow cylinder when pressed on by a syringe or an IV in order to allow the connection of the syringe or the IV onto the proximal female luer connection.

2. The apparatus of claim 1, wherein the valve comprises a luer activated valve or a septum.

3. The apparatus of claim 2, wherein the luer activated valve comprises:
a flexible member having a proximal slit; and
a piston within the flexible member and having a central fluid passageway at the proximal end and one or more radial fluid passageway openings towards the distal end of the piston.

4. The apparatus of claim 1, wherein the proximal female luer connection comprises a luer-lock or a luer-slip.

5. The apparatus of claim 1, wherein the housing further comprises a luer-lock or a luer-slip around the distal male luer tip.

6. The apparatus of claim 1, wherein the cover member is coated with an antibacterial material.

7. The apparatus of claim 1, wherein the proximal female luer connection has an outer surface and the outer surface is coated with an antibacterial material.

8. An apparatus for protecting an input connection of a medical valve comprising:
a substantially hollow cylinder with two or more flaps at a proximal end and an aperture at the distal end;
wherein the aperture is configured to receive a portion of the medical valve so that the two or more flaps are positioned above a connection point of the medical valve; and
wherein the two or more flaps are configured to fold into the substantially hollow cylinder when pressed on by a syringe or an IV in order to allow the connection of the syringe or the IV onto the connection point of the medical valve.

9. The apparatus of claim 8, wherein the medical valve comprises a luer activated valve or a septum.

10. The apparatus of claim 8, wherein the substantially hollow cylinder with two or more flaps is coated with an antibacterial material.

11. The apparatus of claim 8, wherein the proximal female luer connection has an outer surface and the outer surface is coated with an antibacterial material.

12. The apparatus of claim 8, wherein the apparatus is configured to be removably attached to the medical valve.
